(19) United States
(12) Patent Application Publication
(10) Pub. No.: US 2005/0119628 A1
(43) Pub. Date: Jun. 2, 2005

(54) Lavage control apparatus and method of using same

(76) Inventors: Jeanene Sant, Preston, ID (US); Dan McNeil, Tempe, AZ (US)

Correspondence Address: COLLIER SHANNON SCOTT, PLLC, 3050 K STREET, NW, SUITE 400, WASHINGTON, DC, 20007, US

(21) Appl. No.: 10/982,841

(22) Filed: Nov. 8, 2004

Related U.S. Application Data
(60) Provisional application No. 60/517,898, filed on Nov. 7, 2003.

(51) Int. Cl. A61M 31/00

(52) U.S. Cl. 604/275

(57) ABSTRACT

The present invention provides an apparatus and method of use for a control of a lavage procedure. The lavage control apparatus comprises of a housing, where the housing has both a first fluid inlet for receiving a first fluid into a first fluid line and a second fluid inlet for receiving a second fluid into a second fluid line, a fluid control module is located within the housing that connects with both first and said second fluid lines. The fluid control module includes a first control valve for the first fluid line and a second control valve for the second fluid line wherein the first fluid line and the second fluid line merge after both control valves creating a mixed fluid with a predetermined temperature. This outlets the mixed fluid to a speculum for use in directing fluid to a user's colon. The method of administering the apparatus includes adjusting one of the valves to change the temperature of the water that is administered to the colon.

### CROSS-REFERENCE TO RELATED APPLICATIONS

The present invention relates to, and is entitled to the benefit of the earlier filing date and priority of, Application No. 60/517,898, filed on Nov. 7, 2003, which is herein incorporated by reference as if fully set forth.

### FIELD OF INVENTION

The present invention relates to a control apparatus and in particular, a control apparatus with which a therapist administers, or a patient self-administers, a lavage or irrigation treatment.

### BACKGROUND

The cleansing of the large intestine, and specifically the colon of a human subject by lavage techniques is well documented. At the ileocecal valve, partially digested material passes from the small to large intestine and thereafter, through the ascending, transverse, and descending colon to the rectum where it is eliminated. The corrugated, sacculated wall of the colon frequently traps exiting waste material that can subsequently become a source for infection, e.g., colitis, constipation, irritable bowel and occasionally, the nidus for disease processes, e.g., Chrohn's disease, heart burn and diverticulitis.

Lavaging of the colon commonly involves the insertion of a speculum, a product exampled in U.S. patent application Ser. No. 10/465,641, which is herein incorporated in its entirety by reference, a tapered tubular instrument, into the rectum to a precise position within the pelvic colon. The speculum's smaller tube end is positioned within a patient's colon while its larger is attached, outside, to a drain tube through which previously trapped waste from the colon is discharged. An irrigation tube is attached to the speculum's inlet detailing enabling delivery of fluid to the colon. The previously referenced speculum assembly is removeably attached via its irrigation and drain hoses, to the apparatus of this invention, the apparatus aiding the administration of lavage therapy.

The fundamental principal of colon lavage therapy requires that, with the drain line occluded, pressurized irrigation fluid is supplied to a speculum and there-through, to the colon of a human subject: the fluid's temperature, delivery pressure and rate may be adjusted. When the colon has reached its empirically determined, patient procedure-specific fill-point, fluid infusion to the colon is interrupted. Following a chosen dwell-time during which the patient's abdomen may be massaged, the flow of irrigation fluid is restored and the drain line opened allowing any previously trapped waste material within the colon to flow through the speculum and drain line assembly to an appropriate point of disposal.

Colon lavage and irrigation therapies have found society's increasing favor in recent times and as a consequence, a number of product/treatment innovations have occurred within the industry for which patent protection has been granted. The benefit to the therapeutic value of lavage consequent to these innovations has been as varied as the patented ideas, the most frequently cited of which are hereinafter discussed.

Improvements to lavage fundamentals described in U.S. Pat. No. 4,626,239 to Ardizzone include a programmable apparatus having a housing in which hot, cold and drain water conduits are mounted, and to which a flow circuit comprising a plurality of flow sensors and a pressure switch are attached. The pressure-sensing scheme described determines that the rate at which irrigation fluid flows to the colon via a speculum, and at which wastewater is subsequently extracted, is inversely proportional. The circuit facilitates the selection of flow rate, continuous or discontinuous, between pre-established upper and lower boundaries thereby preventing over-pressurization of the colon, a potential problem with this design. When pressure in the colon has reached its upper limit, a valve is activated and waste begins to flow from the colon, and as the pressure normalizes, the process can be repeated. The drain conduit spans the housing's entire width, as it flows towards an appropriate point of disposal. Its progress may be observed through a ‘view window.’

Subsequent to Ardizzone, improvements described in U.S. Pat. No. 4,682,979 to Girouard comprise an apparatus in which irrigation fluid is either gravity or pump-fed to the colon via a rectal nozzle. The gravity option necessitates the elevation of a frequently heavy fluid tank above the treatment plane, while the pump option requires the momentary overriding of valves within the flow circuit. The irrigation fluid is heated before it enters the tank by; 1) the blending of individually valved hot and cold water supplies, 2) an ‘immersion-type’ heater positioned within the tank, or 3) a combination of methods 1&2.

The provision of irrigation fluid of constant temperature and therapy-appropriate delivery rates by means 1-3 is intrinsically problematic. For patient safety, temperature gauges/displays are incorporated within the delivery lines of both sources of heated fluid. As Ardizzone, the product incorporates a drain conduit ‘view window.’

Embodiments described in U.S. Pat. No 4,769,015 to Bloxom, Jr. include a base member to which a container of irrigation fluid and related flow monitoring instrumentation is removably attached. The gravity concept is as Girouard and of similar restricted utility. The externally calibrated container is positioned above the patient, and irrigation fluid flowing there from passes through the instrumentation and thereafter into the subject colon by way of an introducer means. The instrumentation's positioning enables optimal visual control of the system.

Embodiments described in U.S. Pat. No. 4,842,580 to Ouelette relate generally to a speculum product incorporating an obturator, said speculum receiving gravity-fed irrigation fluid, thereby replicating the shortcomings peculiar to all previously described systems.

Embodiments described in U.S. Pat. No. 4,874,363 to Abell include a roll-around structure to which lavage system elements including distinct containers for irrigation and waste-contaminated fluids, a potential health risk, are attached. The flow circuit is similar in principle to Ardizzone, U.S. Pat. No. 4,626,239 but is modified to incorporate a fluid pump, which is operatively connected to a drain line-incorporated pinch valve by electronic, timer-based signals. An air pressure or manually activated switch controls counterpoised functioning (when the pump is on the colon fills, and when the pump is off the colon empties) of drain line pinch valve and pump, and provides constant or pulsed flow alternatives.

Embodiments described in U.S. Pat. No. 5,405,319 also to Abell et al. issued Apr. 11, 1995 include a gravity feed irrigation system as in Girouard. The irrigation flow circuit is derivative of both Ardizzone, and Abell, U.S. Pat. No. 4,874,363 differing from the latter in its utilization of air pressure variation rather than timer-based signals to activate the operatively connected inlet and drain-line valves.

Embodiments described in U.S. Pat. No. 5,788,650 to Dotolo include a housing, flow circuit and associated hardware/principles of use, which are similar to Ardizzone. A unique filter linked in series with a source of ultraviolet radiation, a feature that kills water-borne bacteria and viruses before they enter the colon, is inserted between water pressure valve and hand controlled pressure-limiting switch of the generic flow circuit, which ultimately passing through a supply conduit, terminates in the speculum. Also as in Ardizzone, the drain line from the speculum passes through the housing behind a ‘view window’ as it is flows towards an appropriate point of disposal, and may be detailed to enable sampling of exiting material.

Embodiments described in U.S. Pat. No. 5,019,056 to Lee et al. describe an apparatus which, derivative of Abell, U.S. Pat. No. 4,874,363, comprises a cart configuration to which lavage system elements including distinct containers for irrigation and waste-contaminated fluids, again a potential health risk, are attached. The incorporated flow circuit and associated hardware/principles of use are similar to Abell and to Ardizzone. The generic flow circuit was improved and the irrigation fluid container is now filled with on-board-blended warm water (continuously cooling) that enters the apparatus via flexible hot and cold conduits, removably attached to the domestic supply. A battery powered pump positioned between the outlet of the container and inlet of speculum controls the flow pressure of fluid to the colon, with an associated in-line gauge.

Embodiments described in U.S. Pat. No. 5,190,519 to Mead et al. incorporates distinct containers for irrigation and waste-contaminated fluids and a source of warm irrigation fluid, features conceptually identical to Lee. A unique pumping circuit comprising an auxiliary pump, whose functioning is controlled by a plurality of manually set timers, is positioned between the irrigation container and speculum. It also incorporates a pressure tube positioned on the pump's outlet side, which operatively connected to a pressure port and associated valve (normally closed), 1) returns pumping irrigation fluid to the reservoir if restriction to flow in the colon is sensed by the pressure port, and 2) as Ardizzone, activates the colon draining cycle when the valve is opened at the successful completion of the fill cycle.

Embodiments described in U.S. Pat. No. 5,871,463 to Baker et al. include a portable machine having a unique exterior form in which large vertical scallops are made on left and right sides, enabling the discrete and protected positioning of system inlets/outlets including hot, cold and blended water lines from a domestic source, irrigation outlet and waste return. The incorporated flow circuit with associated hardware/principles of use and, a view window behind which exiting flows towards an appropriate point of disposal, are similar to Abell and to Ardizzone.

Embodiments described in U.S. Pat. No. 5,951,511 to Lowder include individual water tanks that are mounted to a base member as Girouard, at an appropriate height above the treatment plane to insure the gravity feed of irrigation fluid to a patient. A first reservoir receives blended hot & cold water, a second receives cold water, both are constantly replenished by a source of pressurized fluid. Delivery conduits emanating from the two-reservoirs, each incorporating hand operated valves, transition into a single lumen at an intermediate point between reservoir and speculum.

Embodiments described in U.S. Pat. No. 6,106,506, to Abell et al. include a product iterative of an earlier Abell submission, U.S. Pat. No. 4,874,363 having a roll-around structure to which lavage system elements including distinct containers for irrigation, a potential health risk, are attached. The fill/drain scheme is similarly iterative in that a pump draws irrigation fluid from a reservoir through a fill conduit having a sphincter valve integrated at an intermediate positioning along its length to control fluid flow to the speculum inlet, an operatively counterpoised sphincter valve exists between the outlet of the speculum and waste container. The pump of the former iteration functioned as the inlet sphincter valve of the present configuration. Valve and pump performance is computer controlled.

Embodiments described in U.S. Pat. No. 6,228,048, to Robbins comprise a housing in which hot and cold water conduits are mounted and flow circuit together with associated hardware/principles of use are incorporated, similar to Ardizzone. Particular attention is paid in this gravity-fed system to control of temperature and flow pressures, which is implemented by software control.

As experienced lavage therapists, Applicants believe that the improvements described in a significant proportion of the previously cited products would more appropriately be referred to as automations. Colon lavage therapy is by its nature patient centric/patient specific and efforts to promote or codify a universal solution would appear to be contraindicated. It is an advantage of the present invention to provide an apparatus that provides therapeutic assistance to the colon of a human subject, in contradistinction to the irrigation, and only possible cleansing, provided by the previously cited products.

It is an advantage of one but not necessarily all embodiments of the present invention to provide an apparatus that places the control of lavage treatments with the patient and/or therapist; such that the apparatus may adapt to each user's unique and constantly changing therapeutic needs, unlike the cited products.

It is an advantage of one but not necessarily all embodiments of the present invention to provide an apparatus whose requirement for a source of fluid-under-pressure is satisfied by an in-house supply, thus obviating the problems of pump and gravity feed methods. It is an advantage of one but not necessarily all embodiments of the present invention to provide an apparatus that can be readily connected to or disconnected from fluid supply hoses and/or to a facilities existing water-supply.

It is an advantage of one but not necessarily all embodiments of the present invention to provide an apparatus having a toggle-operated hot fluid valve that can terminate hot fluid flow to the system to manually control temperature to assist in protecting the patient and to assist in exercising the colon as it contracts and expands synchronously with alternating streams of hot and cold fluids.

It is a related advantage of one but not necessarily all embodiments of the present invention to provide an apparatus in which the adjustment of cold-fluid flow rate modifies the temperature of fluid within the flow circuit. It is an advantage of one but not necessarily all embodiments of the present invention to provide color-coded fluid lines and corresponding control knobs.

It is an advantage of one but not necessarily all embodiments of the present invention to provide an apparatus having within its flow module a toggle-operated hot fluid flow valve that may function in cooperation with a flow meter that provides irrigation fluid to the speculum at a constant pressure and temperature. The flow meter may be a Watt's type flow meter and may provide fluid to the speculum at a constant pressure and/or temperature.

It is an advantage of one but not necessarily all embodiments of the present invention to provide an apparatus that may incorporate digital panel meters which display both lapsed treatment time and total volume of infused irrigation fluid. The meters may have manufacturer set/ experience-derived limits. It is an advantage of one but not necessarily all embodiments of the present invention to provide an apparatus that is compact, self contained, portable, and is adaptable for use in various user environments.

Additional advantages of various embodiments of the invention are set forth, in part, in the description that follows for an apparatus and method for controlling a lavage procedure.

### SUMMARY OF INVENTION

This invention provides a method and an apparatus for use in lavaging of the colon. In one embodiment of the invention, fluid is supplied to the colon by a lavage control apparatus in which the user is able to control the temperature and the pressure of the fluid supplied to the colon through the use of two control valves that typically are connected to a hot and a cold fluid, although the apparatus could be also envisioned to be used with any two different liquids. The two valves can be used to separately adjust the amount of each fluid that is supplied to the colon. Therefore, the user can adjust the temperature of the fluid supplied to the colon, assuming that the two fluids are not already the same temperature. In one embodiment, one of the valves is a toggle-action valve. A toggle-action valve allows one fluid to be stopped and started instantaneously, which allows for a more immediate temperature fluctuation. In addition, one embodiment of this invention is capable of measuring and displaying both the amount of fluid that is introduced into the colon and the amount of time that it takes to introduce that fluid into the colon. This information is displayed on the apparatus allowing it easily to be recorded by an operator and used to track the process of an individual patient. These embodiments allow the operator of the machine to be capable of administering a wide range of lavage treatments due to the apparatus' ability to adjust to each user's unique and constantly changing therapeutic needs.

In an additional embodiment of the invention, the lavage control apparatus also includes a clean-out capability in which a solution is directed through the waste conduit to assist in the process of keeping the waste conduit clean. In one embodiment, this can be accomplished by using a valve that directs fluid from the lavage control apparatus and blends it with a cleaning fluid and sprays this mixed fluid through the viewable waste conduit and waste outlet to a point of disposal.

In an additional embodiment of the invention, the lavage control apparatus includes a three-way valve to be used to facilitate the transport of the waste product into an acceptable waste disposal. The valve is closed during the filling of the colon, and opened to cease the filling of the colon. When this valve is opened, the waste product is transported through the open three-way valve to an acceptable waste disposal.

It is to be understood that both the foregoing general description and the following detailed description are exemplary and explanatory only, and are not restrictive of the invention, as claimed. The accompanying drawings, which are incorporated herein by reference, and which constitute a part of this specification, illustrate certain embodiments of the invention, and together with the detailed description serve to explain the principles of the present invention.

### BRIEF DESCRIPTION OF DRAWINGS

In order to assist the understanding of this invention, reference will now be made to the appended drawings, in which like reference characters refer to like elements.

FIG. 1 is an isometric view of an apparatus according to an embodiment of the present invention.

FIG. 2 is a front view of an apparatus according to an embodiment of the present invention.

FIG. 3 is an elevational view of an apparatus according to an embodiment of the present invention.

FIG. 4 is an elevational view of an apparatus according to an embodiment of the present invention.

FIG. 5 is a front view of an apparatus according to an embodiment of the present invention.

FIG. 6 is a flow-circuit, hardware schematic of an apparatus according to an embodiment of the present invention.

FIG. 7 is a perspective sketch of a typical connection between a fluid conduit and the source of irrigation fluid under pressure according to an embodiment of the present invention.

FIG. 8 is a top view of the apparatus according to an embodiment of the present invention.

FIG. 9 is an isometric view of the apparatus according to an embodiment of the present invention.

FIG. 10 is a front view of the apparatus according to an embodiment of the present invention.

FIG. 11 is an elevational view of a first side of the apparatus according to an embodiment of the present invention.

FIG. 12 is an elevational view of a second side of the apparatus according to an embodiment of the present invention.

FIG. 13 is a bottom view of the apparatus according to an embodiment of the present invention.

FIG. 14 is a flow-circuit, hardware schematic according to an embodiment of the present invention.

FIG. 15 is a front view of the apparatus according to an embodiment of the present invention.

FIG. 16 is a front view of a speculum of the apparatus according to an embodiment of the present invention.

### DETAILED DESCRIPTION OF EMBODIMENTS OF THE INVENTION

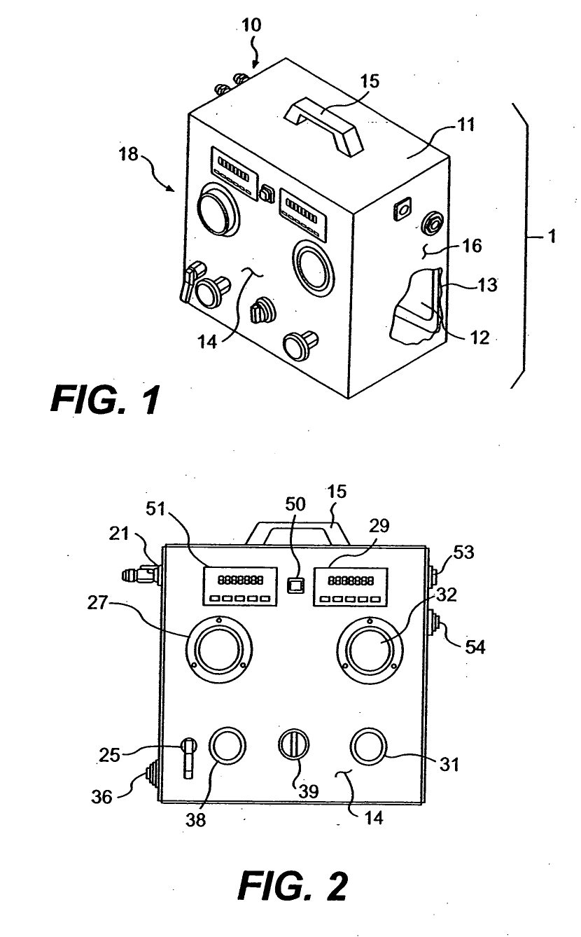

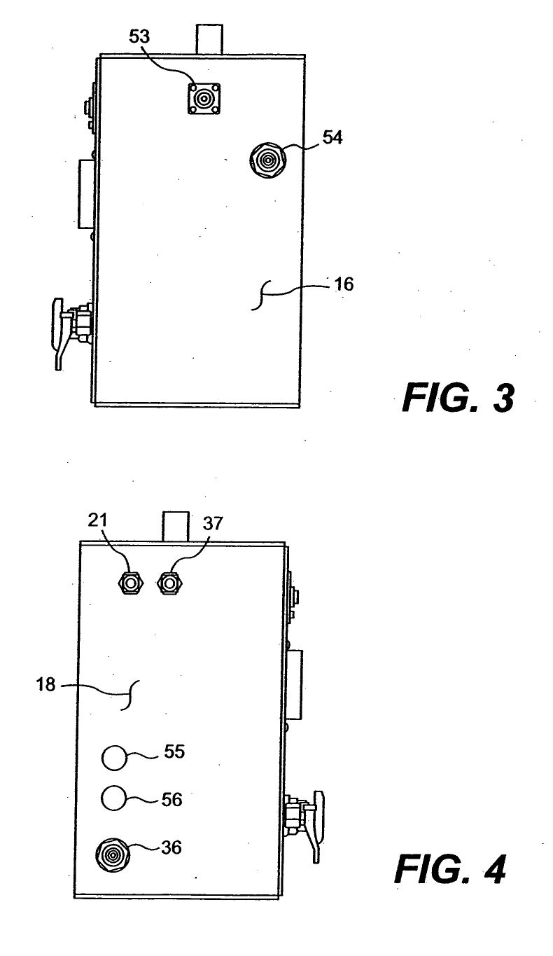

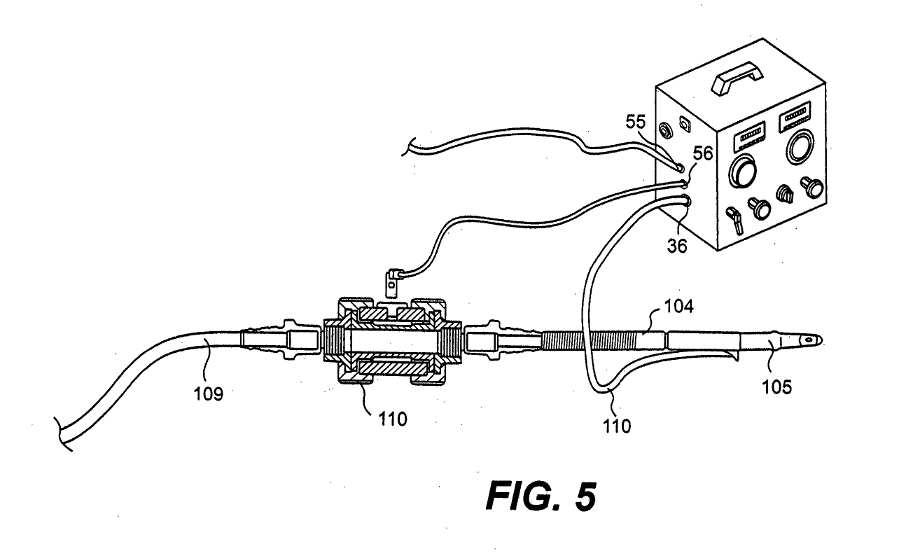

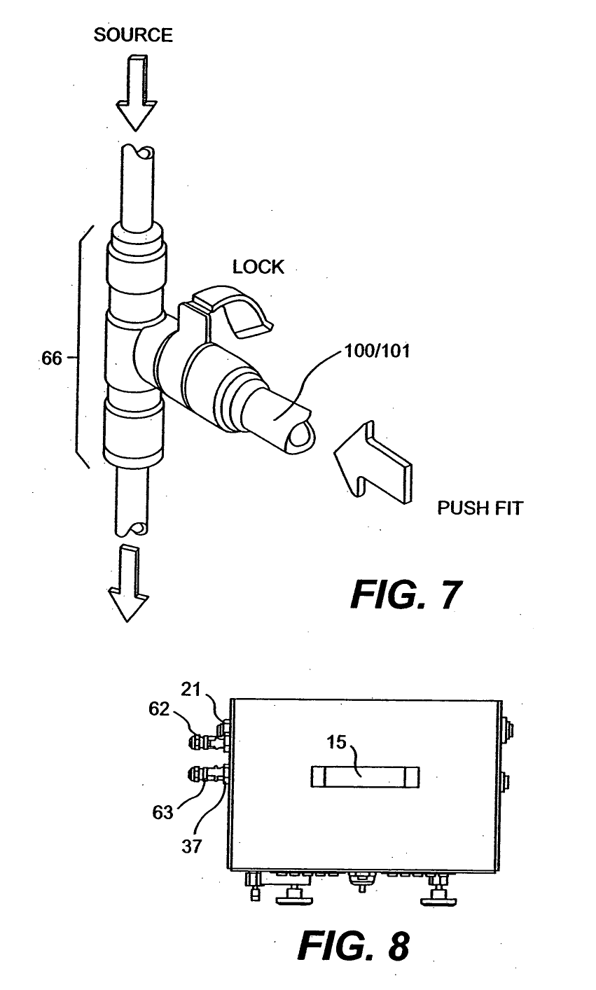

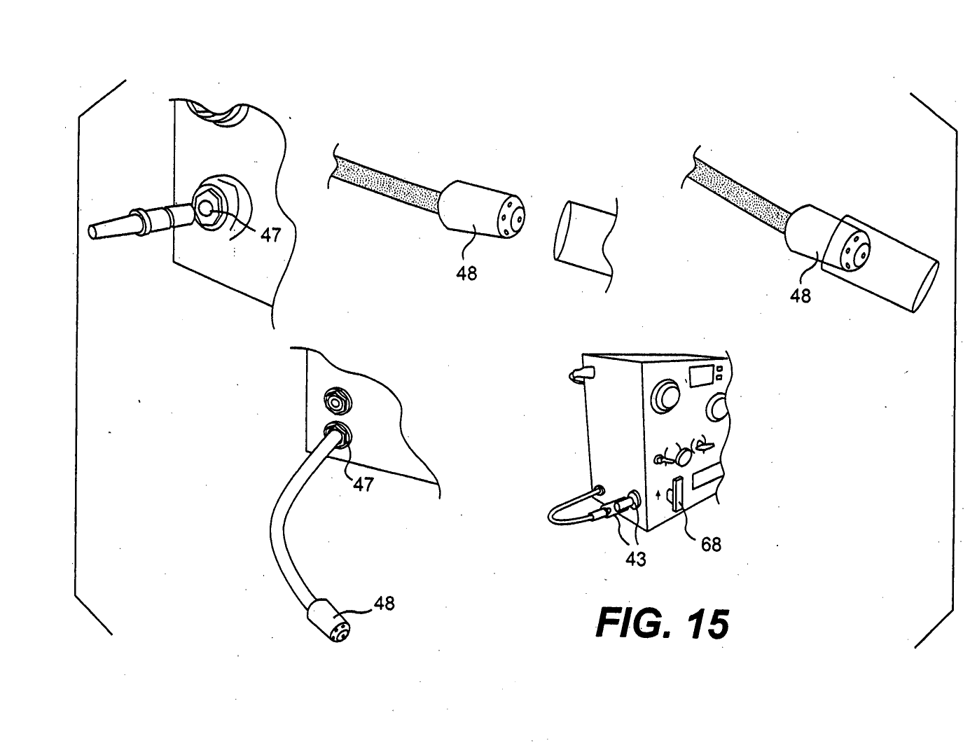

Reference will now be made in detail to embodiments of the present invention, examples of which are illustrated in accompanying FIGS. 1-16. With reference to FIG. 1, an embodiment of the lavage controller apparatus 1 is illustrated. Lavage controller apparatus 1 comprises a molded or otherwise fabricated housing 10, which is manufactured from thermoplastic material, aluminum, or similar lightweight material having appropriate mechanical integrity or any other suitable material. Transport means may be provided by handle 15, which may permanently or temporarily mounted to the housing top 11 or any reasonable means of moving the apparatus 1. An access panel 12, which is positioned in coplanar relationship with the housing's rear surface 13, may be detachably assembled thereto and aids in the product's manufacture and maintenance. Housing 10 front surface comprises a control panel 14 to which manually controlled flow devices, gauges and system-related panel displays, directly linked to the fluid control module 20 (not shown) of apparatus 1, are mounted.

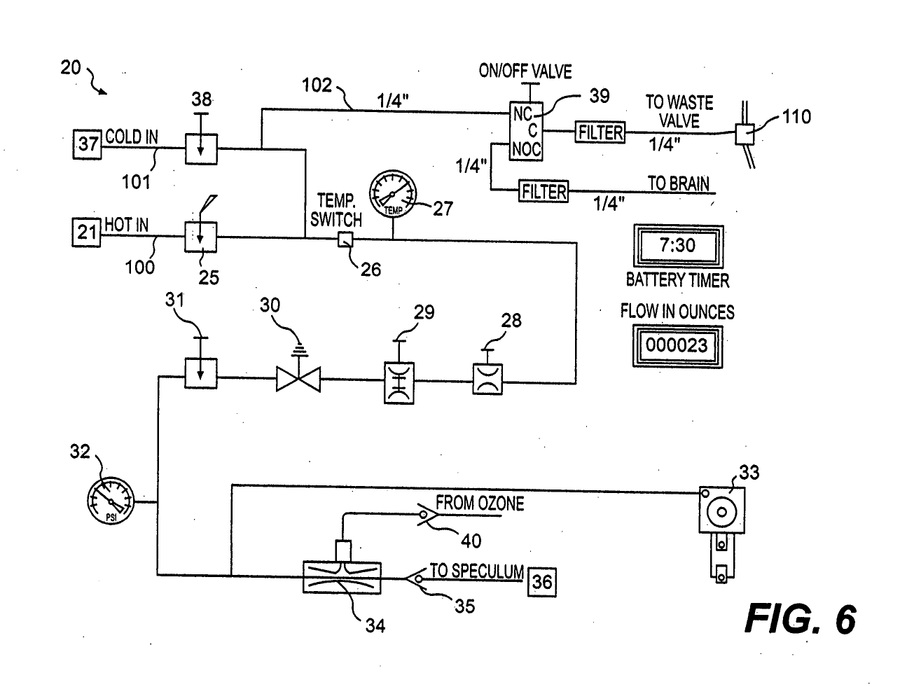

Hardware components together comprising the fluid control module 20 are arranged in functional relationship as illustrated in flow-circuit schematic, FIG. 6 and physically located as defined by the positioning of their related control hardware on the exterior surfaces of the housing 10, as shown in FIG. 2. FIGS. 2, 4, 5, 6, and 8 show bulkhead shut-off couplers 21 and 37 mounted through a housing side wall 18 establishing the attachment point of fluid control module 20 to an external source fluid. Coupler 21 may be, but not necessarily, used to introduce hot or warm fluid into the flow control module 20. Coupler 37 may be, but not necessarily, used to introduce cold or cool fluid into the flow control module 20. As shown in FIG. 6, a first fluid line 100 extends from coupler 21 to a control panel 14 mounted, toggle-action valve 25 then through a temperature switch 26 and temperature gauge 27. Thereafter, the first fluid line 100 passes sequentially through a pressure regulator 28, flow meter 29, solenoid valve 30, a first needle valve 31 and pressure gauge 32 to a pressure switch 33. From this point the first fluid line 100 passes through a DEMA valve 34 (or any other suitable valve) and finally, through a check valve 35 and panel-mounted outlet 36 to the inlet of a speculum. Ozone may be drawn from an appropriate source through a check valve 40 and then blends with fluid flowing through the first DEMA valve 34. The blending may occur by a venturi effect where the ozone is drawn into the DEMA valve 34 due to the pressure difference created by the narrowing of the passage through which fluid in the first fluid line 100 passes.

The temperature switch 26 is set at a preset temperature that the fluid cannot exceed. If this preset temperature is exceeded, a signal is sent to the solenoid valve 30 causing it to close until the temperature of the fluid is below the preset threshold. In one embodiment, this temperature could be set between about 102° F. and about 106° F. However, this maximum temperature threshold could vary depending on the needs of the user. In one embodiment, the pressure switch 33 also ensures that a maximum pressure is not exceeded by the fluid. Typically, the pressure switch is set so that if a pressure greater than about 3 psi is reached at the pressure switch, the solenoid valve 30 will open and close until the pressure of the water is back below about 3 psi. While about 3 psi would be a typical pressure that should not be exceeded for the introduction of water into a colon, this value could be adjusted to accommodate the individual needs of a user.

The second fluid line 101 extends from a second bulkhead coupler 37, and having passed through a valve 38, transitions into the first fluid line 100 at an intermediate positioning between toggle-action valve 25 and temperature switch 26. A third fluid line 102 transitions from the second fluid line 101 between valve 38 and the point where the second fluid line 101 transitions into the first fluid line 100. This third fluid line 102 passes through fill/drain valve 39. When fill/drain valve 39 is open, water passes through valve 39 to a waste valve 110 through waste valve connection 56. In one embodiment, waste valve 110 is a water-pressure-activated pinch valve where the water that passes through fill/drain valve 39 fills a bladder in waste valve 110 so that when the bladder is full, fluid is not capable of flowing through waste valve 110. Waste fluid flowing from the speculum into the waste conduit 104, passes through waste valve 110, which is controlled by fill/drain valve 39. When fill/drain valve 39 is closed, fluid from the third fluid line 102 cannot flow through valve 39 and any fluid inside the bladder in waste valve 110 flows back through fill/drain valve 39 and out to a drain through drain outlet 55. Fill/drain valve 39 may be a manual action switch/3way valve, or any other suitable type of valve. Waste valve 110 may be a water-pressure-activated valve such as a Viton bladder valve, or any other suitable type of valve.

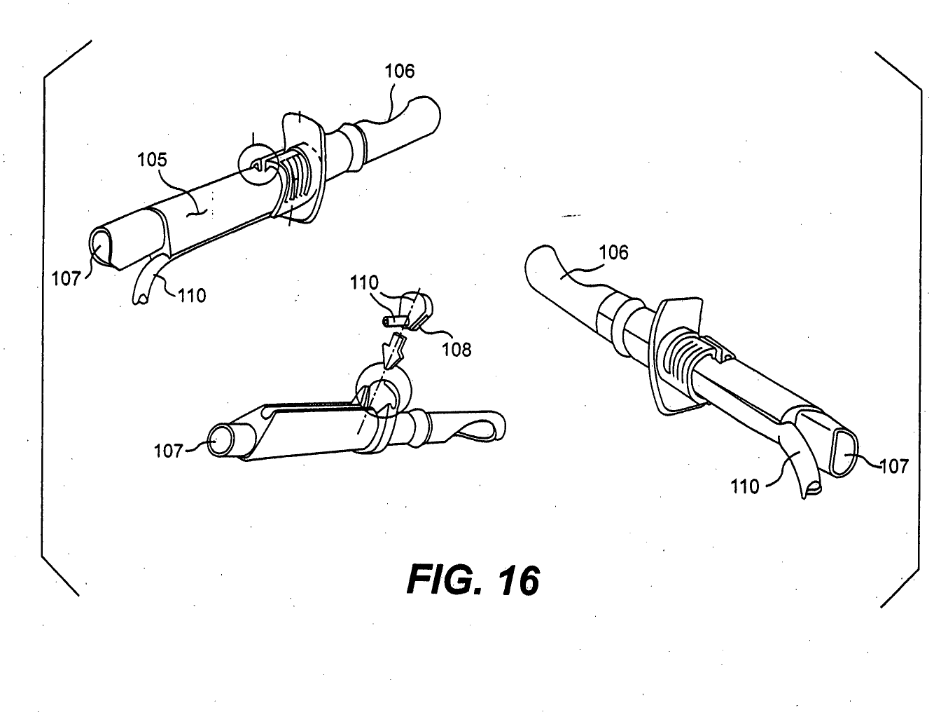

As shown in FIG. 16, speculum 105 has three openings. The first opening 106 is inserted into a user's colon, the second opening 107 is attached to the waste conduit 104, and the third opening 108 receives the fluid from the fluid control module 20 through fluid outlet 36 via tubing 110. When fluid passes through to the speculum 105, it first passes into the waste conduit 104, because typically the second opening 107 is lower than the first opening 106 when the first opening 106 is inserted into the user's colon. Additionally, the fluid will flow through the path of least resistance which would be through the second opening 106 until the waste conduit 104 is full of fluid. When waste valve 110 is in a closed position, fluid is not capable of passing through and collects in the waste conduit 104. When waste conduit 104 is full of fluid and the waste conduit 104 has been entered into the user's colon, fluid is then collected inside the user's colon. Fluid is collected in the user's colon until the desired amount of fluid has been entered. Upon reaching this point, waste valve 110 is opened allowing the fluid in waste conduit 104 to pass through waste valve 110 and to a point of disposal. In one embodiment, a plastic tubing 109 can be attached to waste valve 110 to collect this fluid and direct it to an appropriate point of disposal. Because fluid is no longer being forced into the user's colon, the fluid that is now in the colon will pass out of the colon through the waste conduit 104, go through waste valve 110, and to an appropriate point of disposal through plastic tubing 109. Additionally, fluid from the third opening 108 continues to flow through the second opening 107 to the waste conduit and assists the flow of waste from the first opening 106 as it passes through the waste conduit 104. Plastic tubing 109 typically flows from waste valve 110 via continuous gravity flow. Therefore, waste valve 110 must be a slightly higher level then the opening of the plastic tubing 109 at the point of disposal for waste to properly flow downhill to the point of disposal.

As shown in FIG. 2, control panel 14 may comprise a lever or toggle action first fluid valve 25 and related temperature gauge 27, a knob-controlled second fluid valve 38, a fill/drain valve 39, a needle valve 31 and related pressure gauge 32. A power on/off button 50 may be positioned on control panel 14 and may be located between a timer 51 that measures and displays lapsed treatment time and may have a preset time limit including, but not limited to, about 40 minutes, and a flow meter 29 that measures and displays total volume of fluid infused and may have a preset volume limit, including but not limited to, about 110 fluid ounces. A power inlet 53 and an ozone infusion inlet 54 may be mounted on side panel 16 of housing 10. Shut-off bulkhead quick couplers 21 and 37 receiving a first and second fluid, respectively, from a source of fluid under pressure are mounted on the opposite side panel 18 together with a drain outlet 55, a fluid outlet 36 and a waste valve connection 56. The flow meter 29 that displays the total volume of fluid infused because it allows the operator to calculate the total amount of fluid that has been introduced into the colon each time that an individual uses the apparatus 1. Typically, a user is capable of accepting somewhere between about 30-110 ounces of fluid into their colon. Recordation of the amount accepted each time that the apparatus I is used allows the operator to compare results of the amount of fluid that a user has previously been able to accept into their colon and the amount typically accepted by a person of similar size and weight. This data could provide valuable information regarding the user's colon. Additionally, the timer 51 which measures and displays the amount of time that fluid has been entering the colon can be used by the operator to determine the flow rate of the fluid into the user's colon by reading the amount of fluid from flow meter 29 that enters the colon in a measured amount of time from timer meter 51 . Again, comparison of this result with other results of this user or other user's could provide valuable information regarding the user's colon.

Fluid supply hose components may be reversibly connected to an in-house or portable fluid supply. Each of the fluid lines 100-101 has termination-detailing 62-63 at a first end, complimentary to the housing-mounted bulkhead quick couplers 21 and 37. Both second ends 64-65 of the lines 100-101 may be pressed into an ‘o’ ring sealed/valved branch feature or any other suitable mechanism of a fluid coupling 66 which may be incorporated within the source's plumbing or other fluid source. Incorporation of the coupling 66 is affected by transecting the source conduit and then pressing the resulting tube-ends, one in each of the coupling's 66 through the lumen or by any other suitable mechanism. One advantage to the present system is that it allows the apparatus to adapt to each user's unique and constantly changing therapeutic needs. For example, the present invention allows the toggle-action valve 25 to terminate fluid to the system from the first fluid line 100 instantaneously which assists in protecting the patient. But the toggle-action valve 25 also can be used to exercise the colon by creating an instantaneous temperature change in the fluid causing the colon to either expand or contract. For example, during the draining process, fluid continues to flow through the speculum 105 to the waste conduit 104. Because this fluid is still in close proximity to the colon, a temperature change from hot to cold causes the colon to contract, which assists the draining process by forcing fluid and waste out of the colon. The instantaneous temperature change is achieved by either opening or closing the toggle-action valve 25 when there are fluids of two different temperatures in the first fluid line 100 and the second fluid line 101. A normal rotational valve would only gradually closes, which allows for a substantially less instantaneous temperature change of the fluid. In addition, the operator is also capable of adjusting the temperature using valve 38 (a typically more gradual change in flow) which modifies the temperature of the fluid within the fluid control module 20 when less severe temperature variations are desired. As a means to help identify which lines correspond to which valve, one embodiment of the present invention has color coded lines and valves to assist the operator in determining which line each valve corresponds.

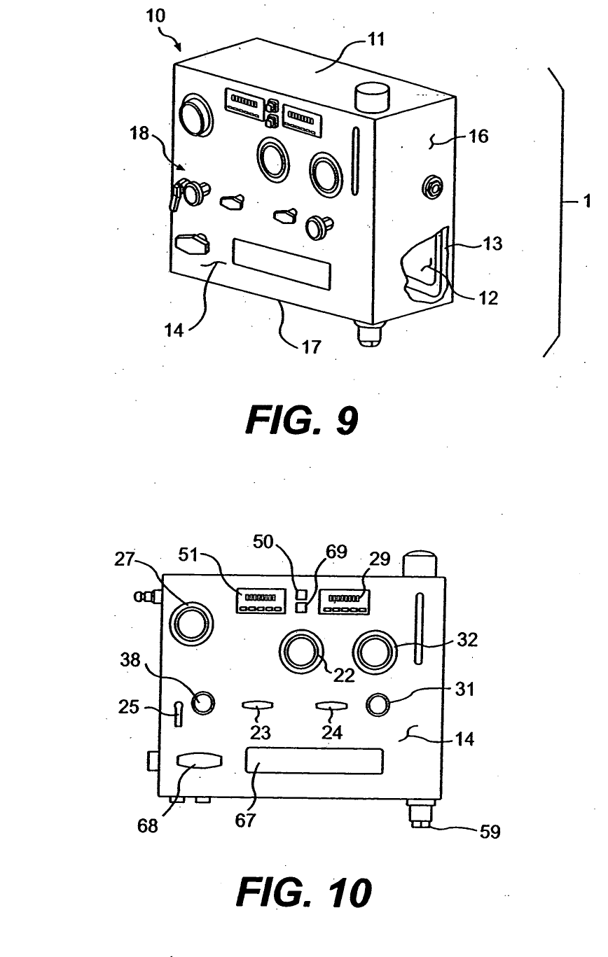

With reference to FIG. 9, an alternative embodiment of a lavage apparatus 1 is illustrated. The lavage apparatus 1 comprises a molded or otherwise fabricated housing 10, which is manufactured from thermoplastic material, aluminum or similar lightweight material having appropriate mechanical integrity or any other suitable material. Transport means may be provided either by a handle detail 15 (not shown) which may be temporarily or permanently mounted to the housing top 11 or, by detailing incorporated therein. An access panel 12 which is positioned in coplanar relationship with the housing's rear surface 13, may be detachably assembled thereto and aids the product's manufacture and maintenance. The housing's front surface comprises a control panel 14 to which manually-controlled flow devices, gauges and system-related panel displays, directly linked to the fluid control module 20 (not shown) of the apparatus 1, are mounted.

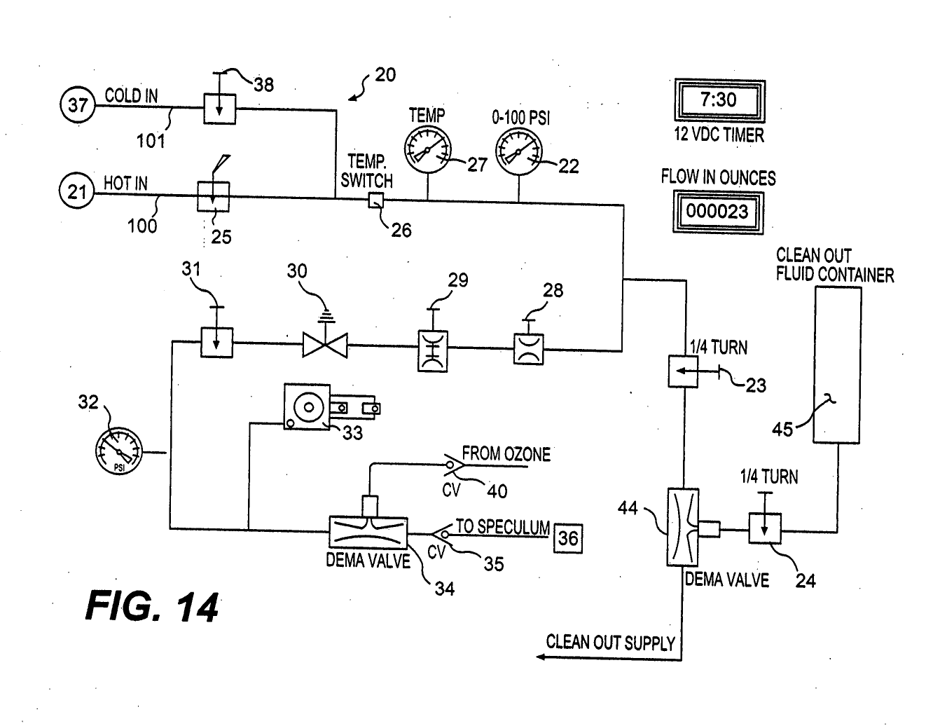

Hardware components together comprising the fluid control module 20 are arranged in functional relationship as illustrated in flow-circuit schematic, FIG. 14, and physically located as defined by the positioning of their related control hardware on the exterior surfaces of the housing 10, as shown in FIG. 10.

As shown in FIGS. 10, 13 and 14, bulkhead shut-off couplers 21 and 37 mounted through the housing base 17 establish the attachment point of fluid control module 20 to external source fluid. The first line extends from coupler 21 to a control panel mounted, toggle-action valve 25 then, through a temperature switch 26, temperature gauge 27 and a second pressure gauge 22. Thereafter the first line passes sequentially through a pressure regulator 28, flow meter 29, solenoid valve 30, a first needle valve 31 and pressure gauge 32 to a pressure switch 33. From this point the first line passes through a DEMA valve 34 and finally, through a check valve 35 and panel-mounted outlet 39 to the inlet of a speculum. Ozone may be drawn from an appropriate source through a check valve 40 and then blends with fluid flowing through the first DEMA valve 34. The blending may occur by a venturi effect. The temperature switch 26 is set at a preset temperature that the fluid cannot exceed. If this preset temperature is exceeded, a signal is sent to the solenoid valve 30 causing it to close until the temperature of the fluid is below the preset threshold. In one embodiment, this temperature could be set between about 102° F. and about 106° F. However, this maximum temperature threshold could vary depending on the needs of the user. In one embodiment, the pressure switch 33 also ensures that a maximum pressure is not exceeded by the fluid as it enters the colon. If a pressure greater than about 3 psi is reached at the pressure switch, the solenoid valve 30 will open and close until the pressure of the water is back below about 3 psi. While about 3 psi would be a typical pressure that should not be exceeded for the introduction of water into a colon, this value could be adjusted to accommodate the individual needs of a user. Coupling 21 may be used to introduce hot or warm fluids, but could be used to introduce any temperature fluid. The second line extends from a second bulkhead coupler 37, and having passed through a valve 38, transitions in to the first line at an intermediate positioning between toggle-action valve 25 and temperature switch 26.

Waste conduit 104 connects to waste inlet 43 which in turn connects to the viewable waste conduit 58 on the front side of the housing 10 allowing the operator to view the contents through the front side of the housing. Preferably, a clear glass or plastic portion of the front side of the housing 10 is used to allow viewing access; however, other transparent embodiments including a hole in the front side of the housing 10 could be used. The viewable waste conduit 58 is then connected to a waste outlet 59 which is used to dispose of the waste, possibly by connecting to a sewer line or any other appropriate waste conduit or housing.

Speculum 105 has three openings. The first opening 106 is inserted into a users colon, the second opening 107 is attached to the waste conduit 104, and the third opening 108 receives the fluid from the fluid control module 20 through fluid outlet 36 via tubing 112. When fluid passes through to the speculum 105, it first passes into the waste conduit 104, because typically the second opening 107 is lower than the first opening 106 when the first opening 106 is inserted into the user's colon. Additionally, the fluid will flow through the path of least resistance which would be through the second opening 106 until the waste conduit 104 is full of fluid. Further, it is advantageous to direct the fluid toward the second opening 107 so that fluid is not directly sprayed on the colon. When drain valve 68 is in a closed position, fluid is not capable of passing through and collects in the waste conduit 104. When waste conduit 104 is full of fluid and the waste conduit 104 has been entered into the user's colon, fluid is then collected inside the user's colon. Fluid is collected in the user's colon until the desired amount of fluid has been entered. Upon reaching this point, drain valve 68 is opened allowing the fluid in waste conduit 104 to pass through drain valve 68 and into the viewable waste conduit 58 and then through to waste outlet 59 which is used to dispose of the fluid waste, possibly by connecting to a sewer line or any other appropriate waste conduit or housing.

In an alternative embodiment, a drain line clean-out capability is added to the flow control module 20. The flow circuit directing irrigation fluid from the first and second lines to the speculum is intersected by a connector at an intermediate positioning between the second pressure gauge 22 and pressure regulator 28. Redirected irrigation fluid at the connector flows through a ¼-turn shut-off valve 23 and a second DEMA valve 44. A clean-out fluid reservoir 45 may also be incorporated within the apparatus housing. A hose attached to reservoir 45 transitions through a cleaning solution valve 24 and transitions into the second DEMA valve 44.

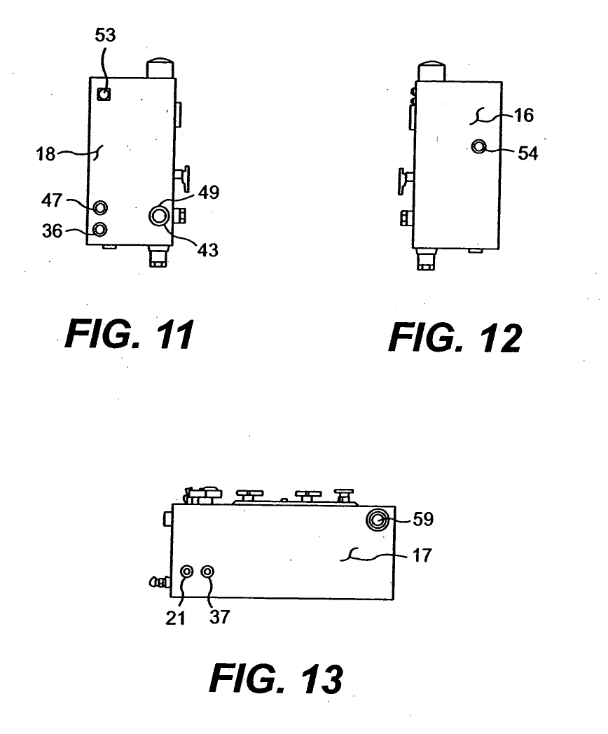

As shown in FIGS. 10 and 11, during a clean-out cycle a flexible conduit 57 having a first end inserted into the fluid-sealed clean-out outlet 47, and a second end adapted to a spray head detail 48, is inserted into short tubular adaptor 49 that in turn is attached by barb detailing to the housing-mounted waste inlet 43. While a spray head detail 48 is used in this embodiment, any method of transferring the fluid from the clean-out outlet 47 including plastic tubing or any other type of tubing that is easily attached and removed could be used. When the clean-out valve 23 is set to the ‘open’ position, irrigation fluid passes through second DEMA valve 44 and ‘clean-out’ outlet 47 to spray head 48 and thereafter, through an open drain valve 68, through viewable waste conduit 58 and waste outlet 59 to a point of disposal (if drain valve 68 is closed, fluid will not be able to pass through to the viewable waste conduit). During a ‘clean-out cycle’ irrigation fluid flows through the ‘viewing chamber’ 67 to a point of disposal. Simultaneously with clean-out solution valve 24 open, clean-out fluid is drawn from reservoir 45 through valve 24 to second DEMA valve 44 where it blends with irrigation fluid flowing to the disposal point. The blend may occur due to a venturi effect or by any other suitable means.

As shown in FIG. 10, the front panel of housing 10 defines the control panel 14 to which a lever-activated first fluid valve 25 and related temperature gauge 27, a knob-controlled second fluid valve 38, a cleaning cycle on/off valve 23, and a second pressure gauge 22 that displays the pressure of source fluid entering the apparatus, are attached. Additionally, a needle valve 31 for controlling the pressure of the outputted fluid and related pressure gauge 27 together with elements of the clean-out system, a fluid level view window and a cleaning fluid supply on/off valve 24 and, the waste view system, a drain valve 68 controlling flow of waste through the housing, a view window 67 and a power on/off switch 69 controlling the illumination of the viewing chamber are present on the control panel.

A power on/off button 50 may be positioned towards the top of front panel 14 and directly beneath chamber light switch 69, and may be located between digital panel meter 51 that records/displays lapsed treatment time and may have a preset time limit, including but not limited to, about 40 minutes, a digital panel meter 52 that records/displays total volume of irrigation fluid infused and may have a preset volume limit, including but not limited to, 110 fluid ounces.

As shown in FIG. 11, a power inlet 53 may be mounted to a first housing side panel 18 together with a fluid outlet 36 (to speculum), cleaning solution outlet 47 and, a waste inlet 43. As shown in FIG. 13, an ozone infusion inlet 54 may be mounted on the opposite side housing 16. As shown in FIG. 13, two female shut-off bulkhead quick couplers 21 and 37, independently receiving a first and a second fluid, respectively, from a source of fluid under pressure are mounted on housing's underside side 17 together with a waste outlet 59.

It will be apparent to those skilled in the arts that various modifications and variations can be made in the construction and configuration of the present invention, without departing from the scope or spirit of the invention. It is intended that the present invention cover the modifications and variations of the invention, provided they come within the scope of the appended claims and their equivalence.

1. A lavage control apparatus, comprising:
a housing, said housing having a first fluid inlet for receiving a first fluid into a first fluid line and a second fluid inlet for receiving a second fluid into a second fluid line,
a fluid control module disposed within said housing in communication with both said first and said second fluid lines, wherein said fluid control module further comprises a first control valve for said first fluid line and a second control valve for said second fluid line wherein said first fluid line and said second fluid line merge after both said first and said second control valves merging said first fluid and said second fluid resulting in a mixed fluid, and
an outlet in communication said fluid control module for supplying said mixed fluid to a speculum for use in directing said mixed fluid to a user's colon.

2. The lavage control apparatus of claim 1, wherein said first control valve is a toggle-action valve.

3. The lavage control apparatus of claim 2, wherein said first fluid and said second fluid have different temperatures.

4. The lavage control apparatus of claim 1, wherein said fluid control module further comprises of a temperature gauge in communication with said mixed fluid which measures and displays the temperature of said mixed fluid.

5. The lavage control apparatus of claim 1, wherein said fluid control module further comprises of a pressure regulator in communication with said mixed fluid.

6. The lavage control apparatus of claim 5, wherein said fluid control module further comprises of a pressure gauge which measures and displays the pressure of said mixed fluid.

7. The lavage control apparatus of claim 1, wherein said fluid control module further comprises of a flow meter which measures and displays the total volume of said mixed fluid directed to said speculum.

8. The lavage control apparatus of claim 1, wherein said fluid control module further comprises of a timer which measures and displays the amount of time fluid has been directed to said speculum.

9. The lavage control apparatus of claim 1, wherein at least one of said first fluid lines is color coded to match said first control valve or said second fluid line is color coded to match said second control valve.

10. A lavage control apparatus, comprising:
a housing, said housing in communication with a first fluid inlet and a second fluid inlet,
a fluid control module disposed within said housing in communication with both said first and said second fluid inlets,
wherein said fluid control module further comprises a first control valve for said first fluid line and a second control valve for said second fluid line wherein said first fluid line and said second fluid line merge after both said first and said second control valves merging said first fluid and said second fluid resulting in a mixed fluid, and
an outlet in communication said fluid control module for supplying said mixed fluid output to a speculum for use in directing said mixed fluid to a user's colon, wherein said mixed fluid is blended with ozone prior to reaching said speculum.

11. The lavage control apparatus of claim 10, wherein said ozone is blended with said mixed fluid using a DEMA valve.

12. The lavage control apparatus of claim 10, wherein said ozone blending occurs by a venturi effect.

13. The lavage control apparatus of claim 10, wherein said fluid control module further comprises of a temperature gauge in communication with said mixed fluid output which measures and displays the temperature of said mixed fluid.

14. The lavage control apparatus of claim 10, wherein said fluid control module further comprises of a pressure regulator in communication with said mixed fluid.

15. The lavage control apparatus of claim 14, wherein said fluid control module further comprises of a pressure gauge which measures and displays the pressure of said mixed fluid.

16. The lavage control apparatus of claim 10, further comprising of a flow meter which measures and displays the total volume of said mixed fluid directed to said speculum.

17. The lavage control apparatus of claim 10, further comprising of a timer which measures and displays the amount of time said mixed fluid has been directed to said speculum.

18. A lavage control cleaning apparatus, comprising:
a housing, said housing having a first fluid inlet for receiving a first fluid into a first fluid line, a second fluid inlet for receiving a second fluid into a second fluid line, and a waste inlet,
a fluid control module disposed within said housing in communication with both said first and said second fluid lines,
wherein said fluid control module further comprises a first control valve for said first fluid line and a second control valve for said second fluid line wherein said first fluid line and said second fluid line merge after both said first and said second control valves merging said first fluid and said second fluid resulting in a mixed fluid,
an outlet in communication said fluid control module for supplying said mixed fluid to a speculum for use in directing fluid to a user's colon,
a waste conduit in communication with said speculum that removably connects to said waste inlet, and
a fluid cleaning line that removably connects to said waste inlet.

19. The lavage cleaning apparatus of claim 18 wherein said fluid cleaning line connects to at least one of said first fluid line or said second fluid line, said cleaning fluid line having a cleaning solution valve that connects said fluid cleaning line to a cleaning solution housing which contains a cleaning fluid.

20. The lavage cleaning apparatus of claim 19 wherein said cleaning solution valve is a DEMA valve.

21. The lavage cleaning apparatus of claim 19 wherein said cleaning solution is pulled into said cleaning solution valve by a venturi effect.

22. A method of administering a lavage treatment by an operator comprising:
using a lavage treatment apparatus comprising of, a housing, said housing having a first fluid inlet for receiving a first fluid into a first fluid line and a second fluid inlet for receiving a second fluid into a second fluid line,
a fluid control module disposed within said housing in communication with both said first and said second fluid lines,
wherein said fluid control module further comprises a first control valve for said first fluid line and a second control valve for said second fluid line wherein said first fluid line and said second fluid line merge after both said first and said second control valves merging said first fluid and said second fluid resulting in a mixed fluid, and
an outlet in communication said fluid control module for supplying said mixed fluid to a speculum for use in directing fluid to a user's colon,
wherein said first valve and said second valve have a first position resulting in a first temperature of said mixed fluid; and
adjusting at least one of said first valve or said second valve to a second position resulting in a second temperature of said mixed fluid.

23. The method of claim 22 wherein said first valve is a toggle-action valve and said adjustment of at least one of said first valve or said second valve includes either opening or closing said toggle-action valve.

24. The method of claim 22 further including monitoring the amount of said mixed fluid entering into said colon using a flow meter.

25. The method of claim 22 further including monitoring the amount of time said mixed fluid enters said colon.

26. The method of claim 22 further including monitoring the rate of flow of said mixed fluid into said colon using a timer and said flow meter
